(19) United States
(12) Plant Patent Application Publication
(10) Pub. No.: US 2003/0014782 P1
(43) Pub. Date: Jan. 16, 2003

(54) Shrub rose plant named 'POULnoev'

(76) Inventors: L. Pernille Olesen, Fredensborg (DK); Mogens N. Olesen, Fredensborg (DK)

Correspondence Address: Poulsen Roser Pacific, Inc., 620 S. Front Street, Central Point, OR, 97502, US

(21) Appl. No.: 10/155,850

(22) Filed: May 24, 2002

Related U.S. Application Data
(63) Continuation of application No. 09/282,071, filed on Mar. 29, 1999.

(51) Int. Cl. A01H 5/00

(52) U.S. Cl. PLT/120

(57) ABSTRACT

A new garden rose plant which has abundant, salmon flowers, attractive foliage, and a compact but vigorous habit. This new and distinct variety has shown to be uniform and stable in the resulting generations from asexual propagation.

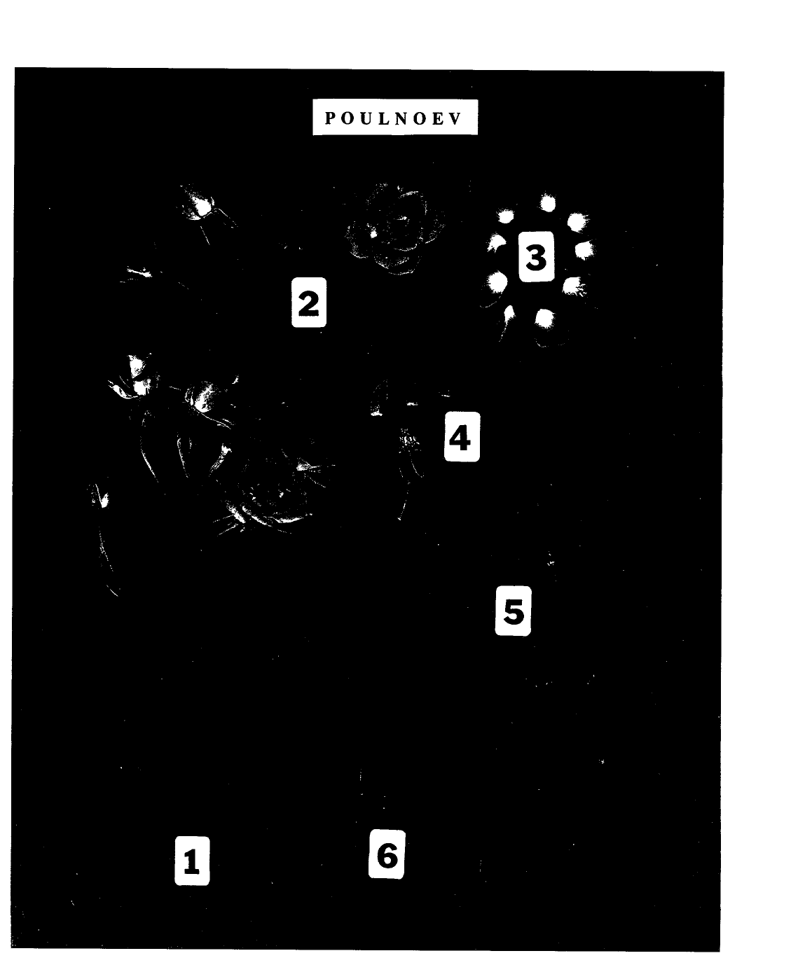

### SUMMARY OF THE INVENTION

The present invention constitutes a new and distinct variety of garden rose plant which originated from a controlled crossing between unnamed seedling (non-patented) and an unnamed seedling (nonpatented). The two parents were crossed and the resulting seeds were planted in a controlled environment. The new variety is named ‘POULnoev’.

The new rose may be distinguished from its seed parent, unnamed seedling, by the following combination of characteristics:

1. The seed parent is a grandiflora with significantly larger flowers when compared to the flowers of ‘POULnoev’;

2. The seed parent's habit is much taller and larger than that of ‘POULnoev’.

3. The seed parent's blooms are bronze in color while those of ‘POULnoev’ are salmon-colored.

The new variety may be distinguished from its pollen parent, unnamed seedling created by the same inventors, by the following combination of characteristics:

1. The pollen parent has orange-red blooms, while ‘POULnoev’ has salmon-colored blooms.

2. The pollen parent has smaller blooms when compared to the blooms of ‘POULnoev’.

The objective of the hybridization of this rose variety for nursery and garden use was to create a new and distinct variety with unique qualities, such as:

1. Uniform and abundant flowers;

2. Vigorous, compact growth;

3. Disease resistance;

4. Bright, salmon-colored blooms.

This combination of qualities is not present in previously available commercial cultivars of this type and distinguish ‘POULnoev’ from all other varieties of which we are aware.

As part of their rose development program, L. Pernille Olesen and Mogens N. Olesen germinated the seeds from the aforementioned hybridization and conducted evaluations on the resulting seedlings in a controlled environment in Fredensborg, Denmark.

‘POULnoev’ was selected in Spring, 1988 by the inventors as a single plant from the progeny of the hybridization.

Asexual reproduction of ‘POULnoev’ by cuttings and traditional budding was first done by L. Pernille and Mogens N. Olesen in August of 1988. This initial and other subsequent propagations conducted in controlled environments have demonstrated that the characteristics of ‘POULnoev’ are true to type and are transmitted from one generation to the next.

### BRIEF DESCRIPTION OF THE DRAWING

The accompanying color illustration shows as true as is reasonably possible to obtain in color photographs of this type, the typical characteristics of the buds, flowers, leaves, stems, and a plant of ‘POULnoev’. Specifically illustrated in SHEET 1:

1. Stem or entire plant showing branching and the attachment of leaves, buds, and peduncles;

2. Flower bud, partially opened bud, and open bloom;

3. Flower petals, detached;

4. Sepals, receptacle, and pedicel;

5. Flowering stem as well as a bare stem exhibiting thorns;

6. Leaves.

### DETAILED DESCRIPTION OF THE VARIETY

The following is a description of ‘POULnoev’, as observed in its outdoor growth in a field nursery in Jackson County, Oreg., on plants aged eighteen months. Observations were conducted during August, 1988. Color references are made using The Royal Horticultural Society (London, England) Colour Chart, 1995, except where common terms of color are used.

For a comparison, several physical characteristics of the rose variety ‘POULrouge’, a miniature rose variety from the same inventors described and illustrated in U.S. Plant Pat. No. 9,687 and issued on Nov. 12, 1996 are compared to ‘POULnoev’ in Chart 1.

CHART 1

| | ‘POULnoev’ | ‘POULrouge’ |
|---|---|---|
| Color of top of | Orange-Red Group 43D to | Red Group 40B. |
| open bloom. | Red Group 40C. | |
| Size of Bloom | 40-45 cm. | 40 mm. |
| (Diameter) | | |
| Petalage | Semi-double, 10-12 petals. | Double, 30-35 petals. |

Parents: unnamed seedling ×unnamed seedling

Classification:

Botanical.—Rosa hybrida.

Commercial.—Miniature.

### FLOWER AND FLOWER BUD

Blooming habit: Recurrent.

Flower bud:

Size.—Upon opening, 22 mm -26 mm in length from base of receptacle to end of bud.

Bud form.—Pointed ovoid.

Bud color.—As sepals unfold and at ¼ opening, Orange-Red Group 33C in mid-zone of reverse side: Towards margin tending towards Orange-Red Group 33B.

Sepals.—Upper side: Yellow-Green Group 144A; lower side: Yellow-Green Group 144A. Many sepals present intonations of Greyed-Red Group 180A on inner and outer surfaces. Moderate foliaceous appendages on three of the five sepals. Surfaces of sepals slightly pubescent. Stipitate glands are present on margins and outermost surface of sepals. Sepals are 13 to 17 mm long and 8 to 10 mm wide. The apex is subulate.

Receptacle.—Surface: Smooth. With white thin hairs. Some rec. with whitish bloom. Shape: Funnel shaped Size: Medium. 5 mm (h) ×5 mm (w). Color: Yellow-Green Group 144A.

Peduncle.—Surface: Smooth. With white thin hairs. Length: 35-45 mm average length. some longer to 60 mm. Color: Yellow-Green Group 144B. Strength: Strong.

Borne.—Generally multiple buds per stem with 15-25 buds per flowering stem.

Flower bloom:

Fragrance.—Little or no fragrance.

Duration.—As a cut flower 2 to 3 days. The blooms have a duration on the plant of approximately 2 to 4 days. Petals fall cleanly away from plant.

Size.—Average flower diameter is 40-45 mm when open.

Form.—Semi-double cup when open but has classic high centered bud form. Shape of flower when viewed from the side: Upon opening, upper part: Convex. Upon opening, lower part: Convex. Open flower, upper part: Convex. Open flower, lower part: Flattened convex.

Petalage.—Semi-double Average range: 10-12 petals under normal conditions with 3-4 petaloids.

Color:

Upon opening, petals:

Outermost petals.—Upper Surface: Middle zone tends toward Orange-Red Group 43D. While marginal zone tends towards Orange-Red Group 43B. Reverse Side: Orange-Red Group 33C.

Innermost petals.—Upper Surface: Orange-Red Group 40D. Reverse Side: Orange-Red group 38A.

Upon opening, basal petal spots: Petals: Outer Side: Yellow Group 2C. Inner Side: Yellow Group 7B.

After opening, petals:

Petals.—Upper Surface: Orange-Red Group 43C with middle zone Red Group 43D, lighter in color then the marginal zone. Upper surface of petal with overtone mixed tone of Orange-Red Group 30A. Which give petal a luminous color. Petals present an occasional, vertical streak of White Group 155 C. Reverse Side: Orange-Red Group 38B with darker margin Orange-Red Group 43C.

Innermost petals.—Upper Surface: Red Group 40C, with an occasional, vertical streak of White Group 155C. Reverse Side: Red Group 38A.

Upon opening. basal petal spots:

Petals.—Outer Side: Yellow Group 2C. Inner Side: Yellow Group 9B.

General Tonality: On open flower Orange-Red Group 33C. No change in the general tonality at the end of the 2nd-3rd day. Afterwards, general tonality is Red Group 33D.

Petals:

Petal reflex.—Petals reflexed, with most being double reflexed, forming a point.

Petal edge.—Entire, with occasional notch.

Shape.—Deltoid to nearly round on some outer petals.

Petaloids.—Quantity: 3-4. Petaloids are 5 to 8 mm wide and 7 to 10 mm long. Petaloid coloration is Red Group 40C.

Thickness.—Average.

Arrangement.—Not imbricated.

Reproductive Organs:

Pistils.—Length: 8 to 12 mm. Quantity: 15 to 18.

Pollen.—Color: Yellow-Orange Group 14B. Abundance: Above average abundance.

Anthers.—Size: 3 to 4 mm long. Color, immature: Greyed-Yellow Group 160D. Color, mature: Greyed-Orange Group 165C-Brown Group 200C Abundance: 17 to 22.

Filaments.—Color: Yellow Group 4A. Length: 5 to 7 mm.

Stigmas.—Much superior in location to anthers. Color: Yellow-Green Group 145D.

Styles.—Color: Green-White Group 157C.

Other intonations.—Immediately below stigma.

Hips.—None observed.

### PLANT

Plant growth: Moderate, upright to bushy. When grown as a budded field grown plant on Rosa multiflora understock, the average height of the plant itself is 75 cm and the average width is 35 cm.

Stems:

Color.—Young wood: Green Group 138A, graduating to Yellow-Green Group 146A. Older wood: Green Group 137C.

Thorns.—Incidence: 0 to 1 per 10 cm of stem.

Surface.—Young wood: Smooth. Older wood: Smooth.

Plant foliage: Quantity of leaflets on normal leaves in middle of the stem: 5-7 leaflets.

Leaf size.—Medium 85-90 mm (1) ×55-60 mm

Abundance.—Average

Color.—Upper Leaf Surface: Yellow-Green Group 147A. Lower Leaf Surface: Yellow-Green Group 146C-147C.

Anthocyanin intonation.—Yes. Location: Underside of leaves, rachis, petioles, stipules and stems. Color: Greyed-Red Group 180A.

Plant leaves and leaflets:

Stipules.—Size: 12 mm -14 mm. Color: Green Group 137B.

Presence of stipitate glands.—On margins of stipules.

Petiole.—Length: 18 mm -22 mm. Color: Green Group 137B. Underneath: Smooth Margins: With stipitate hairs on margins.

Rachis.—Color: Green Group 137B-137C. Underneath: Smooth. Margins: With stipitate glands on margins of rachis and petiolules.

Leaflet.—Edge: Serrated. Shape: Ovate. The leaflet's apex is acuminate. The leaflet's base is rounded. Arrangement: The leaflets are arranged in an odd-pinnate formation. Venation: The leaflets are veined in a reticulate pattern. Texture: Leaves are thick. Upper side of leaflet is glossy. Lower side of leaflet is matte.

Winter hardiness: ‘POULnoev’ has been found to be resistant to damage from cold in USDA Zone 8 and USDA Zone 7.

1. A new and distinct variety of rose plant of the miniature class, substantially as herein illustrated and described as a distinct and novel rose variety due to its abundant, salmon flowers, vigorous growth, disease resistance, and extended period of bloom:
